(19) United States
(12) Patent Application Publication
(10) Pub. No.: US 2026/0174962 A1
(43) Pub. Date: Jun. 25, 2026

(54) METHOD FOR REDUCING INJECTION PAIN OF CERTAIN DRUG FORMULATIONS

(71) Applicant: Ahuva Cices, New York, NY (US)

(72) Inventor: Ahuva Cices, New York, NY (US)

(21) Appl. No.: 19/425,540

(22) Filed: Dec. 18, 2025

Related U.S. Application Data
(60) Provisional application No. 63/737,131, filed on Dec. 20, 2024.

(51) Int. Cl. A61M 5/19; A61K 9/00; A61K 9/19; A61K 38/24; A61K 47/02; A61K 47/10

(52) U.S. Cl. CPC A61M 5/19; A61K 9/0019; A61K 9/19; A61K 38/24; A61K 47/02; A61K 47/10

(57) ABSTRACT

Various embodiments of the present disclosure provide methods and apparatus for reducing pain associated with injection of certain drug formulations. An apparatus as described herein comprises a first chamber or section containing a pharmaceutical formulation comprising menotropin or a variant thereof, a second chamber or section comprising a diluent, and a combining mechanism configured to puncture one or more of the first chamber or the section chamber to combine the pharmaceutical formulation and the diluent prior to injection. A method as described herein comprises actuating a combining mechanism which combines a pharmaceutical formulation comprising menotropin or a variant thereof and a diluent, and actuating an administering means which administers the pharmaceutical formulation and the diluent to a patient.

### CROSS-REFERENCE TO RELATED APPLICATIONS

This application claims the benefit of U.S. Provisional Ser. No. 63/737,131, filed Dec. 20, 2024, the entire contents of which are hereby incorporated by reference in their entirety.

### FIELD OF THE DISCLOSURE

The present disclosure relates to a method for reducing pain associated with injection of certain drug formulations.

### BACKGROUND OF THE DISCLOSURE

Assisted reproductive technology (ART) is a broad field involving the use of various technologies to improve fertility outcomes. One example of the use of ART in clinical settings involves administering gonadotropins to ovulatory patients for purposes of developing multiple follicles and pregnancy. The gonadotropin family of hormones are extensively involved in the fertility cycle in males and females. Gonadotropins can generally be derived from urine, though some (human chorionic gonadotropin, luteinizing hormone, follicle stimulating hormone) can be produced recombinantly. With respect to assisted reproductive technology, a number of gonadotropin hormone-containing drugs are used to stimulate ovulation. FSH is primarily used in these drugs as it is primarily responsible for ovarian stimulation, but they may additionally contain LH and/or hCG as well. Menotropins are a fertility medication that contains high levels of pituitary gonadotropins.

A popular drug that has been used for this purpose for decades is Menopur®, a usually self-administered biologic product comprising menotropins for injection. Specifically, Menopur® is commonly used for the induction of ovulation and assisted reproductive technology. Menopur® contains equal amounts of follicle stimulating hormone (FSH) and human chorionic gonadotropin (hCG)-driven activity, which is known to include luteinizing hormone (LH). The current formulation of Menopur® is dispensed as a single dose vial of 75 international units (IU) with a single dose vial of 2 ml 0.9% sodium chloride and a Q-cap vial adaptor for mixing. Q-cap vial adaptors are needle-free devices that allow a syringe to be connected to a drug vial without a needle. Patients reconstitute lyophilized powder of menotropins in 0.9% sodium chloride and self-administer the solution subcutaneously immediately after reconstitution. See Ferring Pharmaceuticals (2013), Menopur®.

### BRIEF SUMMARY OF THE DISCLOSURE

The present disclosure relates to improving a fertility treatment plan (e.g., egg freezing, in-vitro fertilization, ovulation induction, and the like) via ease of administration through reduction of pain associated with injection of menotropins. The claimed method is for immediate injection of the formulation after mixing gonadotropin with bacteriostatic saline, thereby allowing higher ratios of bacteriostatic saline. The present disclosure is generally directed to methods including diluents comprised of up to 100% bacteriostatic saline as well as an embodiment with 50/50 ratio of bacteriostatic saline and sodium chloride.

Various embodiments of the present disclosure provide methods and apparatus for reducing pain associated with injection of certain drug formulations. An apparatus as described herein comprises a first chamber or section containing a pharmaceutical formulation comprising menotropin or a variant thereof, a second chamber or section comprising a diluent, and a combining mechanism configured to puncture one or more of the first chamber or the section chamber to combine the pharmaceutical formulation and the diluent prior to injection. A method as described herein comprises actuating a combining mechanism which combines a pharmaceutical formulation comprising menotropin or a variant thereof and a diluent, and actuating an administering means which administers the pharmaceutical formulation and the diluent to a patient.

### BRIEF DESCRIPTION OF THE DRAWINGS

FIG. 1 is a flowchart depicting one example of a fertility injection administration method in accordance with at least one embodiment of the present disclosure.

FIG. 2 depicts one example of a medication delivery apparatus in accordance with at least one embodiment of the present invention.

### DETAILED DESCRIPTION

The present disclosure is directed to patient mixing with a new diluent for immediate injection to reduce injection pain, which increases ease of use and patient adherence. Applicant has found that use of bacteriostatic sodium chloride as the diluent for menotropins at the time of injection dramatically reduces the local side effect of burning pain with subcutaneous administration. Reduction of pain was sustained using a 1:1 mixture of preserved and non-preserved sodium chloride. This novel formulation of menotropins diluted with preserved sodium chloride has the potential to dramatically reduce the quality-of-life burden associated with reproductive assisted technology therapies.

Injection site pain with administration is a common side effect of menotropins, listed as a common adverse effect that occurs in more than 2% of patients in the medication's prescribing information. Real world data suggests pain associated with injection of Menopur is far more common than indicated in the prescribing information. For example, a Google search for “menopur” yields top results such as “why is menopur so painful.” See Menopur: Uses, side effects, alternatives & more. GoodRx. (n.d.). https://www. goodrx. com/menopur/what-is. In a pertinent study, 70.1% of patients reported experiencing pain as a result of the injections. See Barrière P, Avril C, Benmahmoud-Zoubir A, Bénard N, Dejager S., Patient perceptions and understanding of treatment instructions for ovarian stimulation during infertility treatment, Reprod Biomed Soc Online. 2019 Oct. 5; 9:37-47. doi: 10.1016/j.rbms.2019.08.003. PMID: 31993512; PMCID: PMC6976931. Many of the references as discussed herein acknowledge injection site pain and burning as a common phenomenon associated with menotropin administration.

Bacteriostatic sodium chloride contains the additive benzyl alcohol. Benzyl alcohol is a bacteriostatic agent with known local anesthetic properties. Use of bacteriostatic sodium chloride has been shown to reduce pain with administration of various medications in the literature. See Alam M, Dover J S, Arndt K A. Pain Associated With Injection of Botulinum A Exotoxin Reconstituted Using Isotonic Sodium Chloride With and Without Preservative: A Double-blind, Randomized Controlled Trial. Arch Dermatol. 2002; 138(4): 510-514. doi: 10.1001/archderm.138.4.510; Hunt S V, Malhotra R. Bacteriostatic preserved saline for pain-free periocular injections: review. Eye (Lond). 2022 Aug; 36(8): 1546-1552. doi: 10.1038/s 41433-021-01925-z. Epub 2022 Jan. 11. PMID: 35017698; PMCID: PMC8749351; Minogue, Sean C. FCARCSI; Sun, Deidre A. FANZCA. Bacteriostatic Saline Containing Benzyl Alcohol Decreases the Pain Associated with the Injection of Propofol. Anesthesia & Analgesia 100(3): p 683-686,March 2005.|DOI:10.1213/01.ANE.0000148617.98716.EB.

It was known to administer FSH and FSH variant formulations comprising benzyl alcohol as a preservative (EP 0974359, “Hoffman”), however, these were prior art solutions addressing a different problem than the present disclosure. Namely, the prior art focused on benzyl alcohol in small quantities for its preservative properties and sought to create more stable liquid formulations for purposes of extending shelf life to allow for multi-use. EP 0974359, “Hoffman”, p. 6. In these formulations, the benzyl alcohol serves as a preservative so the medication can be dispensed to the patient as a liquid that is stable for the duration of a treatment cycle and does not require mixing by the patient for each dose. The prior art teaches that benzyl alcohol and similar preservatives can cause aggregation or degradation of the target proteins. The prior art attempted to solve this problem through reduced ratios of benzyl alcohol to achieve the stated goal of shelf life and stable liquid formulations. See Hoffman at p. 6 (use concentration sufficient to yield an anti-microbial effect, and is readily determined by the skilled artisan, teaching that benzyl alcohol did not adversely affect the biological activity of FSH or a FSH variant and allows multi-use administration at lower ratios of 10 mg/ml benzyl alcohol, which led to 10% minimal dissociation (>50% intact) stored at room temperature for 3 months); see also U.S. Pat. No. 9,463,221 (teaching that FSH with 0.17 mg/ml of benzyl alcohol and heat had loss of structure, and that use of 15 mg/ml benzyl alcohol as a preservative allowed multiple injections over a month, but reported destabilizing effects that increased with increasing concentration); ES2358330T3 (“Samaritani”) (teaching use of FSH or LH liquid with phenol or m-cresol as preservative, including tests of 0.9% benzyl alcohol). Thus, skilled artisans were motivated to reduce the amount of preservatives such as benzyl alcohol to avoid destabilization. See U.S. Pat. No. 11,666,635 (“Sjogren”) (reports preservatives such as benzyl alcohol negatively affect FSH multi dose formulations due to decreased long term stability (decreased temperature of denaturation).

Menotropin and Follicle-Stimulating Hormone (FSH) are distinct (though related) substances used in reproductive medicine. Menotropin is a hormone extracted from the urine of postmenopausal women, comprising a mixture of FH and LH (sometimes occurring in a 1:1 ratio). Menotropin stimulates ovarian follicular growth and development, thereby aiding in ovulation and fertility treatments. Follicle-stimulating hormone (FSH) is a hormone produced by the pituitary gland in both men and women. FSH plays a crucial role in regulating the menstrual cycle and stimulating the growth and maturation of ovarian follicles. Recombinant FSH (rFSH) is produced synthetically through genetic engineering, and is used in assisted reproductive technologies to induce ovulation and treat infertility. Prior art techniques attempt to achieve stabilization of FSH liquids via the addition of pharmaceutically acceptable salts, such as sodium salts or potassium salts, but require that any formulations including a LH component require that the liquids each be stored in a different vial. See U.S. Pat. No. 9,463,221 B2. Thus, the techniques of adding stabilizing components to a liquid formulation of FSH (pharmaceutical salts, etc.) are not additionally directed towards menotropins, given that they require that the LH component of any formulation is kept in a separate vial and thus not combined with the same components.

Thus, the art teaches away from the present disclosure because the prior art sought to achieve shelf stability and would have motivated persons of skill in the art to address the negative effects of degradation by reducing the ratio of benzyl alcohol to the active ingredient. Further, the art teaches that attempts to add components to a liquid FSH formulation require that any LH activity be stored in a separate vial from both the FSH formulation and any added components to said FSH formulation. Thus, the methods would not be applicable to a menotropin formulation including both FSH and LH. Further, the present disclosure solves a different problem, patient pain relief, and proceeds against the teachings of the prior art by increasing the ratio of benzyl alcohol to the active ingredient to achieve the purpose of pain relief.

The novelty of this approach is confirmed by the twenty-plus year-old disclosures of the prior art teaching that benzyl alcohol and bacteriostatic saline could be used with FSH, but the applicant is not aware of any teachings in the art to use benzyl alcohol immediately before injection to reduce pain. This is despite widespread recognition in the field of disclosure that the current fertility treatment regime is painful for 50% of patients (Jonker et al 2021 (Equivalence of stable menopur liquid formulation with a pen and traditional reconstituted powder, using traditional reconstitution of powder 50% of patients had pain with injection)) and pain is the primary concern for patients in selecting optimal mode of administration. See, e.g., De Mesmaeker G, Calles B, Smith J A. Analysis of Nurse and Patient Preferences for Pre-Filled Pen Devices for Self-Injection of Highly Purified Human Menopausal Gonadotropin (HP-hMG, MENOPUR®). Patient Prefer Adherence. 2023 May 17; 17:1281-1292. doi: 10.2147/PPA.S385247. PMID: 37220565; PMCID: PMC10200119. (50% of patients injecting traditional menopur experience pain, patient respondents rank injections being as painless as possible as most important ranked preference). Some existing solutions attempt to reduce the pain associated with intradermal injections using substances such as benzyl alcohol as preservatives (See WO 2004/087071 A2, for example). However, the deficiencies of using benzyl alcohol in a preservative capacity with respect to the formulations as described herein have been discussed above, such that benzyl alcohol's use as a mixture component for its preservative capabilities have been precluded due to its potential to aggregate or degrade target proteins, particularly with respect to LH. Thus, the present disclosure solves a longstanding need to reduce pain, which is a recognized barrier in reproductive medicine patient care. See Mesmaeker et al 2023, for example. Recently, other solutions have attempted to address the pain problem through different injection techniques, such as a pen designed for less painful injections (See Mesmaeker et al 2023) but applicant is unaware of publications citing a change in formulation at the time of injection to reduce pain.

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “example” are used to be examples with no indication of quality level. Terms such as “computing,” “determining,” “generating,” and/or similar words are used herein interchangeably to refer to the creation, modification, or identification of data. Further, “based on,” “based at least in part on,” “based at least on,” “based upon,” and/or similar words are used herein interchangeably in an open-ended manner such that they do not necessarily indicate being based only on or based solely on the referenced element or elements unless so indicated. Like numbers refer to like elements throughout.

Various embodiments of the present disclosure now will be described more fully hereinafter with reference to the accompanying drawings, in which some, but not all embodiments of the disclosure are shown. Indeed, the disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “example” are used to be examples with no indication of quality level. Like numbers refer to like elements throughout.

Embodiments of the present disclosure are directed to a mechanism for improving fertility injection administration by improving ease of administration via reduced pain. In particular, the embodiments as disclosed are directed towards a method for combining two or more fertility solution components immediately prior to administering the solution.

In the ovulation cycle, Follicle Stimulating Hormone (FSH) is secreted by the anterior pituitary to cause stimulation of ovarian follicles. Said follicle subsequently enlarges over the course of 1-2 weeks, and, upon reaching maturity, begins producing an amount of estrogen in excess of a critical threshold. The pituitary gland, upon detecting such a level of estrogen production, releases Luteinizing Hormone (LH), which triggers the release of a mature egg by rupturing the follicle. Stimulating the ovaries in advance of egg retrieval comprises injecting FSH and LH at higher levels in an attempt to trigger the growth and maturity of multiple follicles.

Acknowledging that Follicle Stimulating Hormone occurs in multiple variants (urinary, plasma-derived, recombinant FSH (rFSH)), it should be appreciated that references to FSH herein may correspond to any known variant of FSH. Similarly, it is appreciated that recombinant variants of many gonadotropins (FSH, LH, hCG, and the like) can now be produced. As used herein, instances of gonadotropins in general can include urinary variants, plasma-derived variants, or recombinant variants of the corresponding gonadotropin. For example, uses herein of luteinizing hormone (LH) also encompass uses of recombinant luteinizing hormone (rLH), and so on.

One possible product may be a formulation including menotropin (e.g., FSH and LH) in a first container and a benzyl alcohol formulation in a second container to be mixed at the time of injection. As used herein, the term “container” may refer to any object, receptacle, etc. capable of storing the subject formulations. In at least some embodiments, the containers as described correspond to vials. In at least some embodiments, the containers as described correspond to cartridges. In general, the containers as described may correspond to any object capable of holding the formulation (such as cartridges or syringes) and configured to be administered via an administration device.

The gonadotropin formulations as disclosed herein may correspond to injectable liquid formulations. Formulations can be supplied as a product having one, two, or more pharmaceutical composition(s) including menotropin, FSH, LH, bacteriostatic saline, benzyl alcohol, and/or sodium chlorine in two or more containers to be combined immediately prior to administration. The product/formulations as described herein can be supplied in any appropriate package. For example, a product can contain a number of pre-filled syringes each including menotropin and additional containers comprising a benzyl alcohol formulation to be added to the pre-filled syringes. In another example, the product can contain a number of syringes which are not filled, as well as a set of first containers comprising a menotropin formulation and a set of second containers comprising a benzyl alcohol formulation. In another example, the product can contain a set of containers comprising a menotropin formulation and an additional set of containers comprising a benzyl alcohol formulation, wherein the containers are compatible with a syringe known to a user. In at least some embodiments, such as those including syringes, the syringes may be packaged in a blister package or other means to maintain sterility. The product may additionally include instructions for using the formulations included therein.

In at least some embodiments, the menotropin formulations correspond to lyophilized powders for combination with additional liquid components.

According to a further aspect, the inventive gonadotropin formulation may be provided as a multi-dose preparation. In other words, the product may be provided as a kit comprising at least one container comprising one or more daily doses of the gonadotropin (menotropin, FSH, etc.), or multiple containers, each comprising a different formulation. In some embodiments, an injection pen for multiple injections may be provided, whereby the gonadotropin solution occupies a first container and the benzyl alcohol solution occupies a second container. In at least some embodiments, the instructions may direct a user to mix the contents of the first container with the contents of the second container immediately prior to administering the injection. In at least some embodiments, a cartridge may be provided in which a user can mix the contents of the first container and the contents of the second container.

Injectable formulations can be sterilized by incorporating sterilizing agents, and/or by filtration through a bacterial-retaining filter. In at least some embodiments, sterilization is achieved by forming sterile solid compositions which can be dissolved or dispersed in a sterile injectable medium such as sterile water immediately prior to use. As used herein, the injectable formulations can be supplied in any number of suitable containers, including, but not limited to, a vial, a pre-filled syringe, injection cartridge, and the like.

The terms “pharmaceutical composition” and “pharmaceutical formulation” are used interchangeably herein. The present disclosure is directed in some embodiments to a formulation which exhibits both a reduction in pain at the time of administration and further advantages, such as high shelf-life stability. This has become possible only with the findings of the present disclosure, which shows that particular components enable the advantageous performance and pain reduction with the liquid formulations described herein.

The compositions as described herein can also contain further additives in addition to those already listed, such as, but not limited to, dispersing agents, emulsifying agents, wetting agents, antibacterial agents, antifungal agents, tonicity agents, and/or further preservatives. In some embodiments, such as those where it is desirable to effect prolonged action, an additional goal of the formulation may be to slow the absorption of the gonadotropin(s) with the use of a liquid suspension of amorphous and/or crystalline material with poor water solubility.

As used herein, the term “stability” may refer to the bio-stability of formulations of gonadotropins. Physical instability of a protein formulation may be caused by any conformational change that reduces at least one biological activity of gonadotropin proteins included in the present disclosure. A “stable” formulation or solution is one in which the degree of aggregation, dissociation, modification, loss of biological activity, and such of the proteins therein is controlled within an acceptable range, and does not show an unacceptable increase over a selected time interval that would result in a clinically significant reduction of biologic activity. Examples of methods by which stability may be assessed include, but are not limited to, measurement of a sample's light scattering, absorbance, optical density, inspection of clarity and/or coloration, molecular size determinations, biological activity, and or/by differential scanning calorimetry (DSC). With respect to various observed applications in which bacteriostatic saline was utilized as a diluent, no visible particulates were observed, nor were any color changes measured, thereby indicating a stable formulation.

The claimed product(s) may be provided, for example to a patient or a professional intending to administer the product(s), as dual containers comprising a vial of a menotropin formulation and a second container containing the benzyl alcohol solution. Such a dual container may be reused multiple times, and may suffice for a single or multiple cycles of patient treatment.

The claimed products may be provided to patients indirectly by providing to clinics, pharmacies, or other such facilities and institutions. The menotropin solution in this case may be provided in a larger container (one liter, for example), such that it provides a large reservoir from which a smaller portion of the menotropin solution may be retrieved and transferred into a smaller container and provided to a patient accordingly. Similarly, the benzyl alcohol solution may be provided in a larger container, such that it provides a large reservoir from which a smaller portion of the benzyl alcohol solution may be retrieved and transferred into a smaller container and provided to a patient accordingly.

The claimed products may include packaging material providing conditions under which the product may be used, along with information as required by regulatory bodies. The packaging material may further include instructions for the patient, including instructions for combining the menotropin formulation with the benzyl alcohol solution to form a solution for administration via a syringe.

### Example Medication Delivery Methods

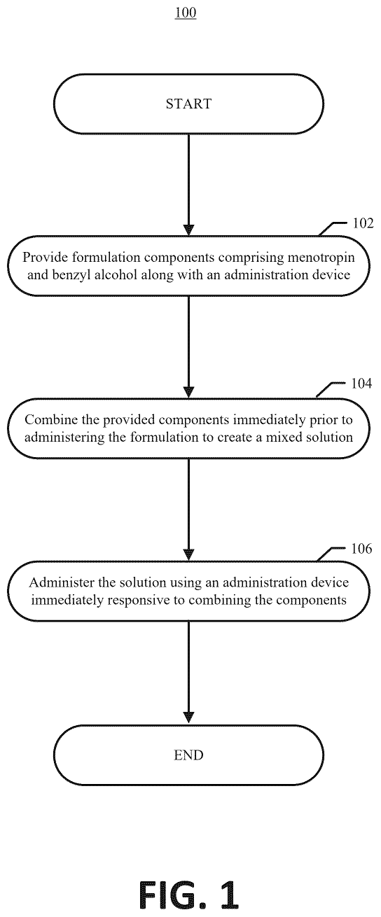

Referring now to FIG. 1, an example method 100 in accordance with some embodiments of the present disclosure is described. As depicted with respect to block 102, the method 100 may include providing injection components comprising menotropin and benzyl alcohol. In some embodiments, preparing injections with benzyl alcohol may include preparing an administration device (e.g., administration needle and corresponding compounds) such that a requisite benzyl alcohol component is stored separately from the other compound components, such as the menotropin formulation. In some embodiments, the method 100 includes preparing one or more containers to be combined at the time of injection. For example, the method 100 may include providing a first container comprising a requisite amount of menotropin formulation, a second container comprising a requisite amount of a sodium chlorine formulation, and a third container comprising a requisite amount of a benzyl alcohol formulation. In other embodiments, the method 100 may include providing a first container comprising a requisite amount of menotropin formulation and a second container comprising a requisite amount of a combination of sodium chlorine and benzyl alcohol. In general, the method 100 may include providing an administration device such that a benzyl alcohol component is stored separately from a menotropin formulation.

As depicted with respect to block 104, the method 100 may include combining the provided components corresponding to the administration device immediately prior to performing the corresponding injection. In at least some embodiments, the method 104 includes combining the contents of a container comprising a requisite amount of a menotropin formulation and the contents of a second container comprising a requisite amount of a benzyl alcohol formulation. In some embodiments, mixing the provided components includes adding a requisite amount of a menotropin formulation as stored in a first container and a requisite amount of a benzyl alcohol formulation as stored in a second container in a third container (such as a syringe, vial, or cartridge, for example) configured for administration via an administration device. In some embodiments, mixing the provided components includes adding a requisite amount of menotropin formulation as stored in a first container to a second container containing a requisite amount of a benzyl alcohol formulation, wherein the second container is configured for administration via an administration device. In some embodiments, mixing the provided components includes adding a requisite amount of a benzyl alcohol formulation as stored in a second container to a first container containing a requisite amount of a menotropin formulation, wherein the first container is configured for administration via an administration device.

As depicted with respect to block 106, the method 100 may include administering the mixed solution to a patient. In some embodiments, administering the mixed solution to a patient includes a patient administering the mixed solution to themselves using a provided needle, syringe, or other administration device. In some embodiments, administering the mixed solution to a patient includes a medical professional administering the mixed solution to a patient using a provided needle, syringe, or other administration device. In some embodiments, administering the mixed solution to a patient generally refers to using an administration device as provided to administer the solution. In yet other embodiments, administering the mixed solution generally refers to filling an administration device previously known to the user or administrator with the mixed solution, and subsequently administering the solution. In at least some embodiments, administering the mixed solution comprises delivering the mixed solution subcutaneously via the administration device.

### Example Medication Delivery Apparatuses

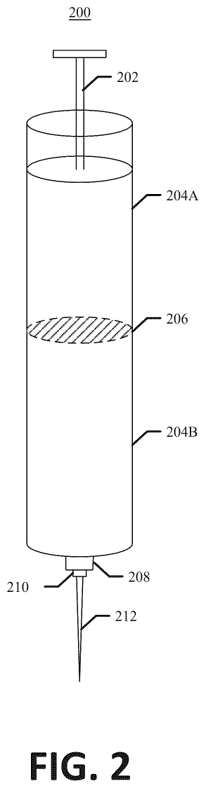

FIG. 2 depicts one example of a medication delivery apparatus 200 in accordance with at least one embodiment of the present invention. As depicted, medication delivery apparatus 200 includes a plunger 202, a first compartment 204A, a second compartment 204B, a divider 206, a needle adapter 208, a needle hub 210, and a needle shaft 212. Medication delivery apparatus 200 may be configured to store a first pharmaceutical component in the first compartment 204A and a second pharmaceutical component in the second compartment 204B such that they will not be combined until administration. It should be appreciated that the medication delivery apparatus 200 is merely one example of an appropriate delivery apparatus corresponding to the techniques as described herein, and does not limit the techniques to such delivery apparatuses/mechanisms. Many additional medication delivery apparatuses may be appropriate for delivering the pharmaceutical formulations as described herein.

Plunger 202 may be a plastic plunger or piston with a rubber tip configured to create a seal between the piston and the barrel. As depicted, the plunger 202 fits tightly within the cylindrical barrel, and can be linearly pulled and pushed along the inside of the barrel, allowing the medication delivery apparatus to take in and expel liquid or gas through a discharge orifice at the front end of the tube. In at least some embodiments, the plunger includes a bottom edge configured to apply pressure enough to breach, break, puncture, or otherwise disrupt a seal (such as divider 206) configured to separate the first compartment 204A and the second compartment 204B.

First compartment 204A and second compartment 204B may correspond to a top half (first half, etc.) and a bottom half (second half, etc.) of a barrel of the medication delivery apparatus 200. In at least some embodiments, the first compartment 204A contains a menotropin solution prior to administration. In such embodiments, the second compartment 204B contains a diluent solution prior to administration. As depicted, the first compartment 204A and the second compartment 204B may be separated by a divider 206. In at least some embodiments, the divider 206 is a breakable seal that can be broken using the plunger 202. In general, the divider 206 may be any seal which can be punctured or otherwise disrupted by applying pressure via the plunger 202 to allow mixture of the components of the first compartment 204A and the second compartment 204B.

Needle adapter 208, needle hub 210, and needle shaft 212 may be specific components of a broader needle component affixed at the end of the medication delivery apparatus 200 configured to administer the pharmaceutical formulation to a patient. Needle adapter 208 (or needle connector) is a device that connects the needle shaft 212, via needle hub 210, to the body of the medication delivery apparatus 200 at the end of the barrel. Example needle adapters include Luer adapters or H transfer connectors. Needle hub 210 corresponds to the end of the needle shaft 212 which connects the needle to the body of the medication delivery apparatus 200, and is configured to connect to the specific needle adapter 208 present. Needle shaft 212 is the hollow elongated part of a needle that connects to the needle adapter 208 via the needle adapter 210, and which penetrates the skin of a patient.

In other embodiments (not depicted), the barrel of the medication delivery apparatus may contain a single compartment comprising either the menotropin solution or the diluent solution. In such embodiments, the medication delivery apparatus may have a detachable end such that the component not stored within the barrel, either the menotropin solution or the diluent solution, can be added to the barrel immediately prior to administration. Further yet, the medication delivery apparatus may be configured such that one component of the pharmaceutical formulation may be stored within the barrel, while the other may be drawn from a separate vial through the syringe, thereby enabling the formulation to be mixed immediately prior to administration.

In at least some embodiments, a provided product includes components for creating multiple doses of the subject menotropin solution. For example, the provided product may include enough menotropin formulation and diluent solution for creating multiple doses of a menotropin solution for administration. In some embodiments, the provided product may include an amount of each component enabling creation of a plurality of identical doses. For example, with respect to a first embodiment of the present disclosure, the mixed solution for injection comprises 2 milliliters of bacteriostatic saline for each menotropin injection. In such embodiments, a provided product intended for multiple injections may include an amount of bacteriostatic saline such that 2 milliliters are available for each injection.

In at least some embodiments, the provided medication delivery device is configured such that combination of the of the menotropin formulation and the diluent solution will occur according via a user operating the medication delivery device as they would a standard syringe. In other words, the provided medication delivery device is configured such that the user need not interact with the solution and the menotropin formulation directly, thereby providing increased usability and ease of use while also eliminating opportunities by which user interaction with the solution(s) could introduce bacteria or other contaminants.

One or more embodiments of the present disclosure are directed towards a menotropin formulation comprising menotropin and bacteriostatic saline. In such embodiments, the bacteriostatic saline may be presence in an amount of 2 milliliters. Optionally, these solutions and formulations may additionally contain a selected buffer and a salt. Such formulations may be provided in a packaging providing enough of the subject formulation for multiple injections, such that each injection will contain the same amount of menotropin and bacteriostatic saline.

One or more embodiments of the present disclosure are directed towards a menotropin formulation comprising menotropin, bacteriostatic saline, and non-preserved saline. In such embodiments, the bacteriostatic saline and non-preserved saline may be present in a 1:1 ratio to one another; for example, the formulation may comprise 1 milliliter of bacteriostatic saline and 1milliliter of non-preserved saline. Optionally, these solutions and formulations may additionally contain a selected buffer and a salt. Such formulations may be provided in a packaging providing enough of the subject formulation for multiple injections, such that each injection will contain the same amount of menotropin, non-preserved saline, and bacteriostatic saline.

One or more embodiments of the present disclosure are directed towards a menotropin formulation comprising menotropin, bacteriostatic saline, and non-preserved saline, such that each component of the formulation is present in varying amounts with respect to varying doses of said formulation. For example, a first dose of the formulation may comprise menotropin, 0.5milliliters of bacteriostatic saline, and 0.5 milliliters of non-preserved saline, and a second dose of the formulation may comprise menotropin and 1 milliliter of bacteriostatic saline. It should be appreciated that bacteriostatic saline and non-preserved saline may be present in varying amounts with respect to various doses of the subject formulation.

One or more embodiments of the present disclosure are directed towards a menotropin formulation comprising 75 units of menotropin (in lyophilized powder form, for example) per vial and 1 milliliter of diluent. In some embodiments, such as those where a 150 unit dose of menotropin is provided in two vials, 1 milliliter of diluent may be added to a first vial containing 75 units of menotropin, after which the resultant mixture may be transferred to a second vial comprising an additional 75 units of menotropin.

### Effects on Injection Discomfort

Various menotropin formulations as discussed above were utilized with respect to test subjects to analyzed and determine the accompanying injection discomfort effects. During observation, pain scores were assessed using a numerical rating scale (NRS) ranging from 0 (no pain) to 10 (most severe pain imaginable). Subjects recorded a first set of pain scores associated with menotropin formulations reconstituted with normal saline, and a second set of pain scores using variations of the novel menotropin formulations as described herein. Each set of test subjects as described herein comprised one healthy adult volunteer undergoing elective egg freezing and had completed one prior cycle of egg freezing using known methods of administration.

A first set of test subjects received a menotropin formulation prepared with 50% bacteriostatic saline or 100% bacteriostatic saline. The first set of test subjects recorded an average NRS score of 9 with respect to the administered injections of formulations reconstituted with normal saline. Using the novel test formulation prepared with 50% or 100% bacteriostatic saline, the first set of test subjects recorded an average NRS score of 1. No local or systemic adverse effects were observed in the first set of test subjects, and participant reported improvement in general well-being during the egg freezing cycle due to the use of the novel formulation.

A second set of test subjects received formulations prepared with 100% bacteriostatic saline. The second set of test subjects recorded an average NRS score of 7 with respect to the administered injections of formulations reconstituted with normal saline. Using the novel test formulation prepared with 100% bacteriostatic saline, the second set of test subjects recorded an average NRS score of 2. No local or systemic adverse effects were observed in the second set of test subjects, and participants reported improvement in general well-being during the egg freezing cycle due to the use of the novel formulation.

Participants unanimously agreed that the novel method of preparation would be preferable if future egg cycles were necessary. On average, the test subjects reported an average reduction of approximately 81% with respect to the pain/discomfort experienced. Further, no adverse effects were noted throughout the treatment cycle including egg retrieval and one-month follow up appointment(s). No irregularities or complications were noted throughout the egg freezing cycle or the retrieval. Both the first set of test subjects and the second set of test subjects completed therapy absent deviations from the expected clinical response, and retrieved more eggs using the novel method of preparation than during a prior egg retrieval cycle following known methods of administration.

What is claimed is:

1. A medical device, comprising:
a first chamber or section containing a pharmaceutical formulation comprising menotropin or a variant thereof;
a second chamber or section comprising a diluent; and
a combining mechanism configured to puncture one or more of the first chamber or the section chamber to combine the pharmaceutical formulation and the diluent prior to injection.

2. The medical device of claim 1, wherein the diluent comprises benzyl alcohol.

3. The medical device of claim 2, wherein the diluent is a combination of bacteriostatic saline and sodium chloride.

4. The medical device of claim 3, wherein the diluent comprises bacteriostatic saline and sodium chloride present in a 1:1 ratio to one another.

5. The medical device of claim 1, wherein the menotropin is initially present as a lyophilized powder prior to combination.

6. The medical device of claim 1, wherein the menotropin is initially present as a pellet prior to combination.

7. The medical device of claim 1, wherein the diluent is bacteriostatic saline.

8. The medical device of claim 1, further comprising an administering means for administering the pharmaceutical formulation and the diluent to a patient.

9. The medical device of claim 8, wherein the administering means comprises one or more of a plunging mechanism and a needle.

10. A method for administering a pharmaceutical formulation, the method comprising:
actuating a combining mechanism which combines a pharmaceutical formulation comprising menotropin or a variant thereof and a diluent; and
actuating an administering means which administers the pharmaceutical formulation and the diluent to a patient.

11. The method of claim 10, wherein the diluent is bacteriostatic saline.

12. The method of claim 10, wherein the diluent is a combination of bacteriostatic saline and sodium chloride.

13. The method of claim 12, wherein the diluent comprises bacteriostatic saline and sodium chloride present in a 1:1 ratio to one another.

14. The method of claim 10, wherein the menotropin is initially present as a lyophilized powder prior to combination.

15. The method of claim 10, wherein the menotropin is initially present as a pellet prior to combination.

16. The method of claim 10, further wherein the pharmaceutical formulation is administered subcutaneously.

17. The method of claim 10, further wherein the pharmaceutical formulation includes 75units of a menotropin component and 1 milliliter of a diluent component comprising benzyl alcohol.
